(19) United States
(12) Patent Application Publication
(10) Pub. No.: US 2004/0215301 A1
(43) Pub. Date: Oct. 28, 2004

(54) Medical lead with a pivotal tip

(76) Inventors: Nicolaas M. Lokhoff, Kerkrade (NL); Paulus G. Adams, Munstergeleen (NL); Ron Van Der Kruk, Bunde (NL); Koen Michels, Maastricht (NL); Jean J.Q. Rutten, Bocholtz (NL)

Correspondence Address: MEDTRONIC, INC., 710 MEDTRONIC PARKWAY NE, MS-LC340, MINNEAPOLIS, MN, 55432-5604, US

(21) Appl. No.: 10/422,067

(22) Filed: Apr. 23, 2003

(51) Int. Cl. A61N 1/05

(52) U.S. Cl. 607/116

(57) ABSTRACT

The invention is directed to a medical lead that includes a pivot element. The pivot element is disposed between a distal electrode and the body of the lead so that the distal electrode can move laterally relative to the lead body. In various embodiments, the pivot element can comprise a hinge element, a double-hinge element, a ball joint element, or the like. The distal electrode may comprise a helical element that can be implanted in tissue. Following implantation of the electrode, the pivot element can reduce stress on tissue surrounding the implantation site, and can decrease the chance of dislodgement of the electrode.

### FIELD OF THE INVENTION

The invention relates to medical devices and, more particularly, to implantable medical leads for use with implantable medical devices (TMDs).

### BACKGROUND OF THE INVENTION

In the medical field, implantable leads are used with a wide variety of medical devices. For example, implantable leads are commonly used to form part of implantable cardiac pacemaker systems that provide therapeutic stimulation to the heart by delivering pacing, cardioversion or defibrillation shocks. The pulses can be delivered to the heart via electrodes disposed on the leads, e.g., typically near distal ends of the leads. In that case, the leads can position the electrodes with respect to various cardiac locations so that the pacemaker can deliver pulses to the appropriate locations. Leads are also used for sensing purposes, or both sensing and stimulation purposes.

In addition, implantable leads are used with neurological devices such as deep-brain stimulation devices, and spinal cord stimulation devices. For example, leads can be stereotactically probed into the brain to position electrodes for deep brain stimulation. Leads are also used with a wide variety of other medical devices including, for example, devices that provide muscular stimulation therapy, devices that sense chemical conditions in a patient's blood, gastric system stimulators, implantable nerve stimulators, implantable lower colon stimulators, e.g., in graciloplasty applications, implantable drug or beneficial agent dispensers or pumps, implantable cardiac signal loops or other types of recorders or monitors, implantable gene therapy delivery devices, implantable incontinence prevention or monitoring devices, implantable insulin pumps or monitoring devices, implantable hearing restoration devices, and the like. In short, medical leads can be used for sensing purposes, stimulation purposes, drug delivery, and the like.

A number of challenges exist with respect to medical leads. In particular, new and improved lead designs are often needed to facilitate medical implantation to specific locations within a patient. For example, as more advanced and complex pacing techniques are developed, it becomes desirable to facilitate lead implantation at new cardiac locations. Some recent advancements in pacing have made use of non-conventional locations for delivery of pacing pulses, such as left ventricular locations, atrial roof locations, inter-ventricular septum locations, and epicardium locations to name a few. Other locations for delivery of therapeutic pulses to the heart or other body locations will likely be discovered and used in the future.

### BRIEF SUMMARY OF THE INVENTION

In general, the invention is directed to a medical lead that includes a pivot element. The pivot element is disposed between a distal electrode and the body of the lead so that the distal electrode can move laterally relative to the lead body. The distal electrode may comprise a helical element that can be implanted in tissue for fixation of the lead. Following implantation of the electrode, the pivot element can reduce stress on tissue surrounding the implantation and decrease the chance of dislodgement of the fixed electrode. Also, the pivot element can reduce stress on the lead and specially on an implanted helical electrode. The invention may be particularly useful for electrode implantation in epicardial locations, and implantation in patients that have relatively weak tissue, e.g., children, infants, fetuses, the elderly, and the like.

In one embodiment, the invention provides a medical lead comprising a lead body that extends from a proximal end to a distal end, a pivot element coupled to the distal end of the lead body, and an electrode coupled to the pivot element.

In another embodiment, the invention provides a system comprising an implantable medical device and an implantable lead. The implantable lead includes a lead body that extends from a proximal end to a distal end, the proximal end being coupled to the implantable medical device. The implantable lead also includes a pivot element coupled to the distal end of the lead body, and an electrode coupled to the pivot element.

In another embodiment, the invention provides a system comprising a medical lead including a lead body that extends from a proximal end to a distal end, a pivot element coupled to the distal end of the lead body, and an electrode coupled to the pivot element, wherein the lead body and the pivot element are formed to define a lumen through the medical lead. The system also includes a stylet inserted through the lumen of the lead body and the pivot element to inhibit pivoting of the pivot element.

In another embodiment, the invention provides a method comprising inserting a stylet through a lumen of a medical lead to inhibit pivoting of a pivot element of the medical lead, implanting a distal electrode of the medical lead in tissue, and removing the stylet to allow pivoting of the pivot element of the medical lead.

### BRIEF DESCRIPTION OF THE DRAWINGS

FIG. 1 is a perspective view of an exemplary implantable medical device system according to an embodiment of the invention.

FIG. 2 is a cross-sectional side view of a distal region of an implantable medical lead according to an embodiment of the invention.

FIG. 3 is another cross-sectional side view of a distal region of an implantable medical lead according to an embodiment of the invention.

FIG. 4 is another cross-sectional side view of a distal region of an implantable medical lead according to an embodiment of the invention.

FIG. 5 is another cross-sectional side view of a distal region of an implantable medical lead according to an embodiment of the invention.

FIG. 6 is a cross-sectional view of two different medical leads implanted in a human heart according to embodiments of the invention.

FIGS. 7 is a conceptual cross-sectional side view illustrating a lead system including a medical lead and a stylet that can be used during implantation of the medical lead.

FIG. 8 is a conceptual cross-sectional side views illustrating the medical lead illustrated in FIG. 7, following removal of the stylet.

FIG. 9 is a flow diagram illustrating a procedure for implanting a medial lead according to an embodiment of the invention.

### DETAILED DESCRIPTION OF THE INVENTION

The invention is directed to a medical lead that includes a pivot element. The pivot element is disposed between a distal electrode and the body of the lead so that the distal electrode can move laterally relative to the lead body. In various embodiments, the pivot element can comprise a hinge element, a double-hinge element, a ball joint element, or the like. The distal electrode may comprise a helical element that can be implanted in tissue for fixation of the lead. Following implantation of the electrode, the pivot element can reduce stress on tissue surrounding the implantation site, and can decrease the chance of dislodgement of the electrode. Also, the pivot element can reduce stress on the lead and specially on an implanted helical element. The invention can be used in any scenario where implantation of an electrode within tissue is desirable. The invention is particularly useful for electrode implantation in epicardial locations, and implantation in patients that have relatively weak tissue, e.g., children, infants, fetuses, the elderly, and the like.

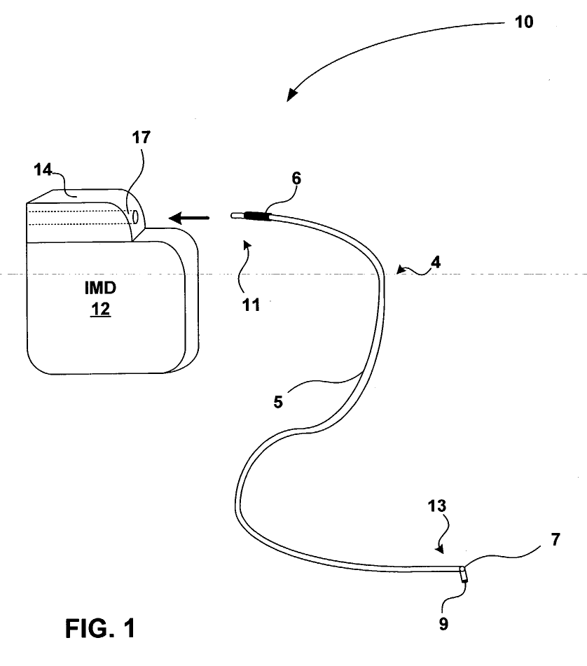

FIG. 1 is a perspective view of an exemplary implantable medical device system 10 for implantation in a human or other living being. In general, system 10 comprises an implantable medical device (IMD) 12, and an implantable medical lead 4 electrically coupled to IMD 12. In some cases, a plurality of additional leads can also be coupled to IMD 12. Implantable medical lead 4 defines a lead body 5 that extends from a proximal end 11 to a distal end 13. In general, implantable medical lead 4 positions electrode 9 within a patient so that therapeutic stimulation pulses can be delivered by IMD 12 to electrode 9 via lead 4. Additional leads and additional electrodes per lead can also be used.

In accordance with the invention, lead 4 includes a pivot element 7 that can reduce stress forces within tissue following implantation of electrode 9 within the tissue. In particular, pivot element 7 allows electrode 9 to move substantially freely relative to lead body 5. Thus, following implantation of electrode 9 within tissue, pivot element 7 can reduce the stress and lateral forces that lead 4 causes on the tissue that surrounds electrode 9.

Although implantable medical lead 4 is illustrated as including a single electrode 9 positioned on distal end 13 of implantable lead 4, any number of additional electrodes (not shown) may be distributed along the length of lead 4. For example, lead 4 may comprise a bipolar lead that includes two electrodes, or may include any number of electrodes along the body of lead 4. Electrode 9 (or other electrodes) can be used for sensing, delivery of stimulation pulses, or possibly the delivery of high voltage shocks to a patient. Also, an integrated sensor element may be disposed along the body of lead 4.

Proximal end 11 of lead 4 includes an electrical contact element 6 which is electrically coupled to electrode 9 via one or more conductive elements that extend through lead body 5. For example, the conductive elements that extend through lead body 5 may comprise coiled filars that form a lumen through lead body 5. Proximal end 11 of lead 4 can be inserted into channel 17 of connector module 14 such that electrical contact element 6 is electrically coupled to circuitry within IMD 12. Connector module 14 forms part of IMD 12 and may be electrically coupled to sensing circuitry and/or stimulation circuitry within IMD 12.

Electrode 9 as well as other electrodes (if desired) can be made from an electrically conductive, biocompatible material such as elgiloy, platinum, platinum-iridium, platinum-iridium oxide, sintered platinum powder or other residue product after combustion with some high heat source, platinum coated with titanium-nitride, pyrolytic carbon, or the like. Although a single lead 4 is shown for purposes of illustration, any number of leads may be used, and thus coupled to connector module 14 of IMD 12.

Electrode 9 may form the distal most region of lead 4 and/or may form a substantially cylindrical ring of conductive material that extends about an exterior wall of lead 4. For example, electrode 9 can extend the entire 360 degrees about lead 4 or to some lesser extent.

In some embodiments, lead 4 can be tubular but not necessarily cylindrical. For example, electrode 9 and lead 4 can have alternative cross sections, e.g., square, rectangular, hexagonal, oval or the like.

In other examples, electrode 9 may assume a shape that can improve or facilitate implantation of electrode 9 within tissue. For example, electrode 9 may comprise a helical element that can pierce and screw into tissue. In that case, the helical electrode element can be guided by the lead to the proper implantation site, and then screwed into the tissue of the patient to anchor the helical electrode within tissue. These or any other electrode configuration could be implemented in accordance with the invention. In any case, following implantation of electrode 9, pivot element 7 of lead 4 allows the implanted position of electrode 9 to rotate laterally relative to lead body 5. Accordingly, movement of lead body 5 within the patient is less likely to dislodge electrode 9 from the implanted location.

IMD system 10 may comprise any system that makes use of an IMD 12 and one or more implantable medical leads 4. For example, IMD 12 can take the form of an implantable cardiac pacemaker, cardioverter or defibrillator, or the like. In some embodiments, IMD 12 may be external (not implanted), with lead 4 forming an implantable portion of system 10. In most cases, however, IMD 12 and lead 4 are both implanted within a patient.

In the description that follows, many details of the invention will be provided in the context of a cardiac pacemaker system. In that case, IMD 12 takes the form of an implantable cardiac pacemaker device that provides therapeutic stimulation to a patient's heart. In other embodiments, IMD 12 can take the form of an implantable cardioverter, an implantable defibrillator, or an implantable cardiac pacemaker-cardioverter-defibrillator (PCD). IMD 12 can deliver pacing, cardioversion or defibrillation pulses to a patient via various electrodes (not shown) disposed along the lead body 5 of lead 4. Accordingly, lead 4 positions one or more electrodes, including electrode 9, with respect to cardiac locations so that IMD 12 can deliver therapeutic pulses to the locations.

The invention, however, is not necessarily limited for use in pacemakers, cardioverters of defibrillators. Other uses of the leads described herein include uses in patient monitoring devices, or devices that integrate monitoring and stimulation features. In those cases, the leads can include sensors disposed on distal ends of the respective lead for sensing patient conditions.

Also, the leads described herein may find use with a neurological device such as a deep-brain stimulation device or a spinal cord stimulation device. In those cases, the leads can be stereotactically probed into the brain to position electrodes for deep brain stimulation, or into the spine for spinal stimulation. In other applications, the leads described herein can provide muscular stimulation therapy, gastric system stimulation, nerve stimulation, lower colon stimulation, drug or beneficial agent dispensing, recording or monitoring, gene therapy, or the like. In short, the leads described herein can find useful applications in a wide variety medical device systems. Specifically, the lead designs described in greater detail below can be very useful in applications where lead 4 is implanted for placement of electrode 9 within tissue.

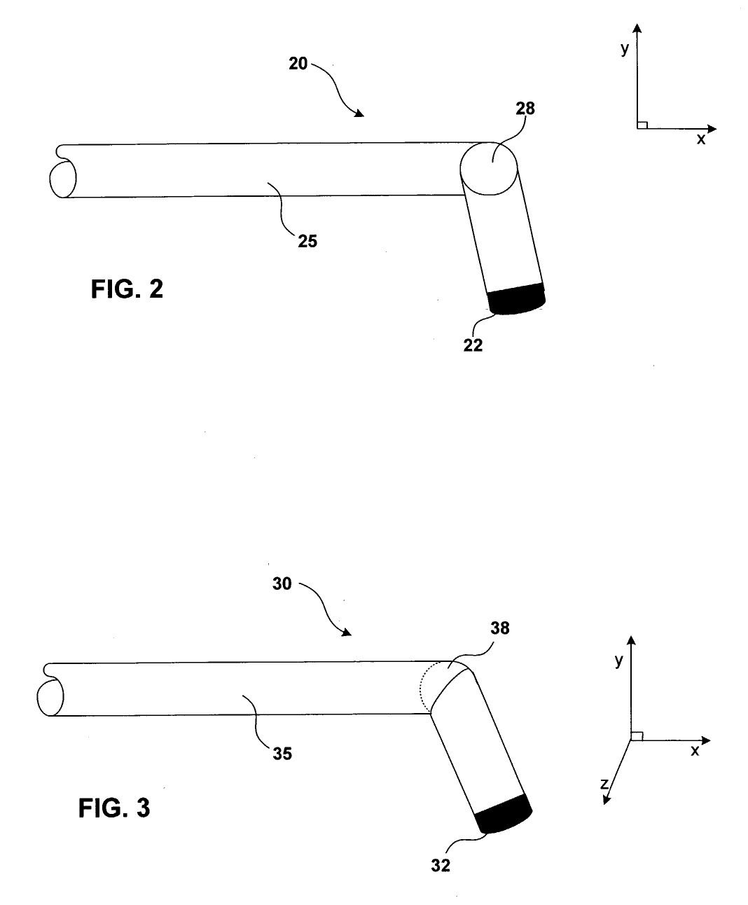

FIG. 2 is a cross-sectional side view of a distal region of lead 20 according to an embodiment of the invention. Lead 20 may correspond to lead 4 of FIG. 1. As shown in FIG. 2, lead 20 includes an electrode 22 disposed on the distal most tip of lead 20. Electrode 22 is electrically coupled to the proximal end (not shown) of lead 20 via one or more conductive elements that extend through the body 25 of lead 20. For example, the conductive elements typically comprise coiled filars that form a lumen through lead body 25. The diameter of lead 20 may be between 1 and 5 millimeters, although the invention is not limited in that respect. Small diameter leads, e.g., less than 2 millimeters, may be particularly useful for pediatric and fetal applications.

In accordance with the invention, lead 20 includes a pivot element 28. In particular, pivot element 28 couples electrode 22 to lead body 25 and allows lateral arc-like motion of electrode 22 relative to lead body 25. In the example, of FIG. 2, pivot element 28 comprises a hinge element that allows electrode 22 to rotate in the x-y plane, as illustrated. In other words, pivot element 28 allows electrode 22 to rotate in two dimensions relative to lead body 25. In one example, pivot element 28 allows for rotation of electrode 22 towards the y-axis relative to the x-axis (where the x-axis corresponds to the axis defined by lead body 25). Such rotation of electrode 22 may span between 110 and −110 degrees relative to lead body 25.

FIG. 3 a cross-sectional side view of a distal region of lead 30 according to another embodiment of the invention. Lead 30 may correspond to lead 4 of FIG. 1. As shown in FIG. 3, lead 30 includes an electrode 32 disposed on the distal most tip of lead 30. Electrode 32 is electrically coupled to the proximal end (not shown) of lead 30 via one or more conductive elements that extend through the body 35 of lead 30. The conductive elements typically comprise coiled filars that form a lumen through lead body 35.

Lead 30 includes a pivot element 38 that couples electrode 32 to lead body 35 and allows for three-dimensional arc-like motion of electrode 32 relative to lead body 35. In the example, of FIG. 3, pivot element 38 comprises a ball joint element that allows electrode 32 to rotate in any direction in x-y-z space, as illustrated. In other words, pivot element 38 allows electrode 32 to rotate in three dimensions relative to lead body 35. In one example, pivot element 38 allows for rotation of electrode 32 in any direction relative to the x-axis (where the x-axis corresponds to the axis defined by lead body 35). Such rotation of electrode 32 may span 110 degrees in any direction from the x-axis defined by lead body 35.

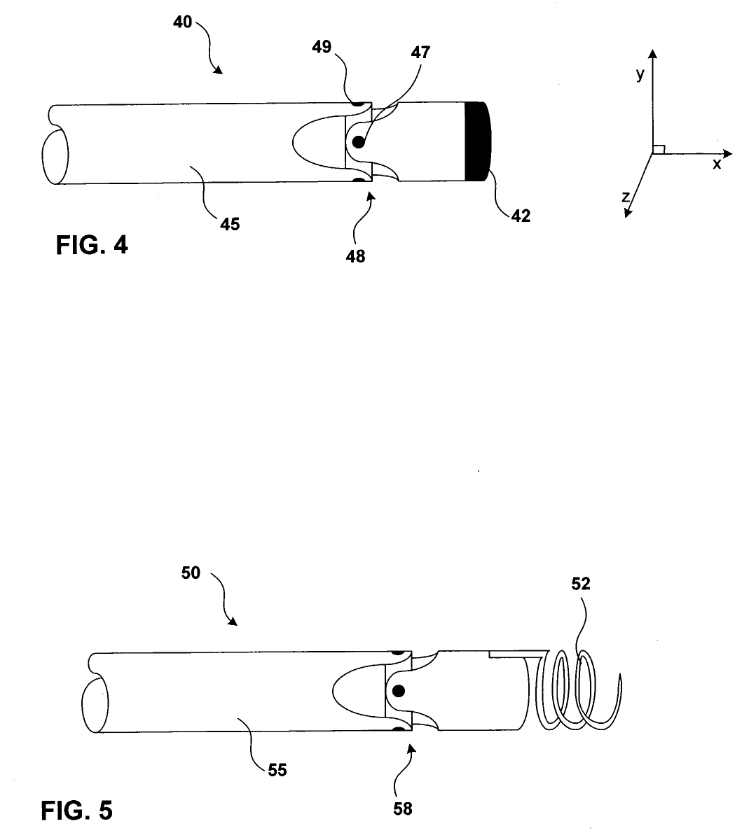

FIG. 4 a cross-sectional side view of a distal region of lead 40 according to another embodiment of the invention. Lead 40 may correspond to lead 4 of FIG. 1. As shown in FIG. 4, lead 40 includes an electrode 42 disposed on the distal most tip of lead 40. Electrode 42 is electrically coupled to the proximal end (not shown) of lead 40 via one or more conductive elements that extend through the body 45 of lead 40. Again, the conductive elements typically comprise coiled filars that form a lumen through lead body 45.

Lead 40 includes a pivot element 48 that couples electrode 42 to lead body 45 and allows for three-dimensional arc-like motion of electrode 42 relative to lead body 45. In the example, of FIG. 4, pivot element 48 comprises a double-hinge element that allows electrode 42 to rotate in any direction in x-y-z space, as illustrated. In other words, pivot element 48 allows electrode 42 to rotate in three dimensions relative to lead body 45. A first hinge 47 allows for hinged rotation relative to the x-y plane, and a second hinge 49 allows for hinged rotation relative to the x-z plane. Accordingly pivot element 48 allows for three-dimensional arc-like motion of electrode 42 in any direction relative to lead body 45. In particular, pivot element 48 allows for rotation of electrode 42 in any direction relative to the x-axis (where the x-axis corresponds to the axis defined by lead body 45). Such rotation of electrode 42 may span 110 degrees in any direction from the x-axis defined by lead body 45.

FIG. 5 a cross-sectional side view of a distal region of lead 50 according to another embodiment of the invention. Lead 50 may correspond to lead 4 of FIG. 1 and in many respects is substantially similar to lead 40 of FIG. 4. For example, lead 50 includes an electrode 52 disposed on the distal most tip of lead 50, which is electrically coupled to the proximal end (not shown) of lead 50 via one or more conductive elements that extend through the body 55 of lead 50.

In the example of FIG. 5, however, electrode 52 comprises a helical element designed for fixation to tissue. For example, a physician can rotate lead 50 to cause helical electrode 52 to screw into tissue an anchor electrode in the tissue. Such a helical electrode may also be used as the respective distal electrode of leads 20 or 30 of FIGS. 2 and 3. In general, however, the distal electrode of leads according to embodiments of the invention can assume any of a wide variety of electrode configurations.

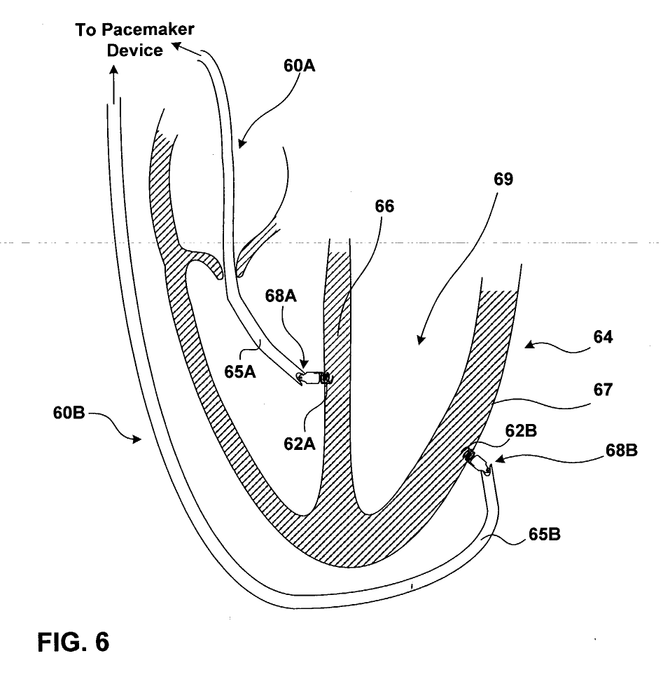

FIG. 6 is a cross-sectional view of medical leads 60A and 60B implanted in human heart 64 according to an embodiment of the invention. In particular, lead 60A is implanted in the inter-ventricular septum 66 of heart 64, and lead 60B is implanted in the epicardium 67 of heart 64 adjacent the left ventricle 69. Leads 60A, 60B respectively include pivot elements 68A, 68B that respectively couple electrodes 62A, 62B to lead body 65A, 65B and allows for rotation of electrodes 62A, 62B to lead bodies 65A, 65B. In the example of FIG. 6, pivot elements 68A, 68B comprises double-hinge elements and electrodes 62A, 62B comprise helical electrodes that can be screwed into tissue.

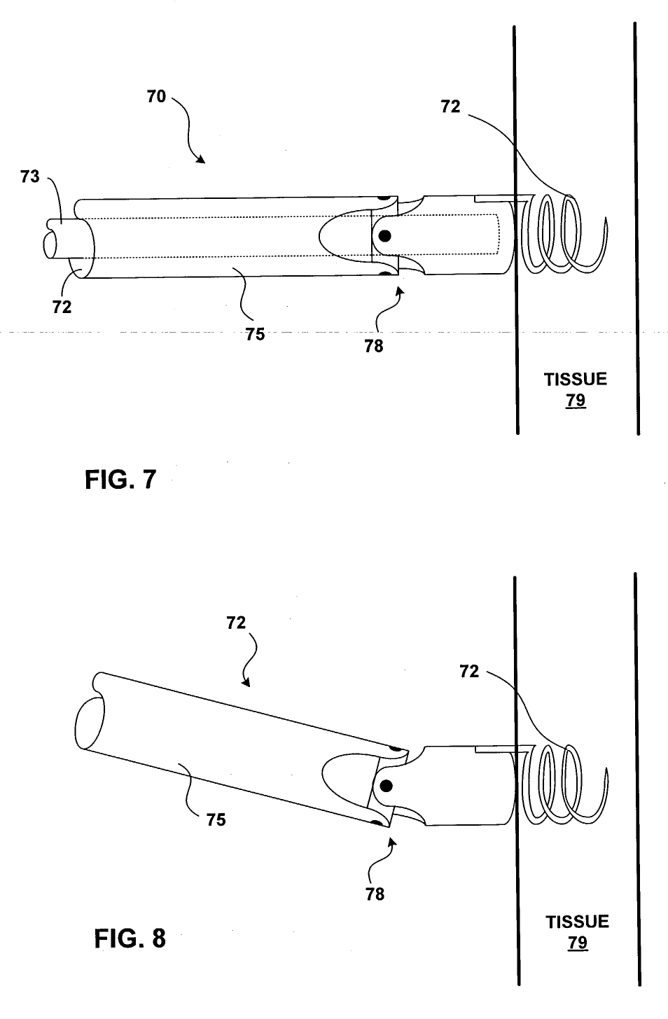

FIGS. 7 and 8 are conceptual cross-sectional side views illustrating a lead implantation system 70 that can be used for implantation of a medical lead 72 into tissue such as the inter-ventricular septum of the heart, the epicardium of the heart, or any cardiac or non-cardiac location where implantation of an electrode is desirable. The lead implantation system 70 includes medical lead 72, and a stylet 73 inserted through a lumen of medical lead 72. Specifically, lead body 75 and pivot element 78 can be formed with the lumen. Insertion of stylet 73 within the lumen of lead 72 can stabilize pivot element 78 and inhibit movement or lateral motion of electrode 71 relative to lead body 75. A physician can guide lead 72 to the proper implant location, possibly making use of a guiding catheter.

Once electrode 71 is positioned adjacent tissue 79 corresponding to the desired implant location, electrode 71 can be implanted in tissue 79, e.g., by rotating lead 72 to screw and anchor electrode 71 in tissue 79. Again, stylet 73 inserted within the lumen of lead 72 can stabilize pivot element 78 and inhibit movement or rotation of electrode 71 relative to lead body 75. In other words, pivot element is generally not allowed to pivot because of the presence of stylet 73 within the lumen of lead 72

Once electrode 71 is properly implanted in tissue 79, stylet 73 can be removed, as shown in FIG. 8. Moreover, once stylet 73 is removed, pivot element 78 allows lead 72 to naturally assume a configuration of reduced stress relative to leads that do not include such a pivot element 78. Specifically, electrode 71 is rotated along an arc, relative to the major axis of lead body 75. Accordingly, stress at the interface of electrode 71 and tissue 79 can be reduced, and the likelihood of dislodgement of electrode 71 from tissue 79 can be likewise reduced. If patient movement causes movement of lead 72, pivot element 78 adjusts for such motion in order to reduce or eliminate stress to tissue 79.

Accordingly, the invention can be particularly useful for electrode implantations in epicardial locations where significant bending of electrode 71 relative to lead body 75 is common. In that case, stress on the tissue as a result of lead bending can be significantly reduced by implementing leads that include one or more pivot elements. Also, the invention can be very useful for implantations in patients that have relatively weak tissue, e.g., children, infants, fetuses, the elderly, and the like. The invention, however, is not limited to such applications, and can reduce stress on tissue in any of a wide variety of cardiac and non-cardiac implantation locations.

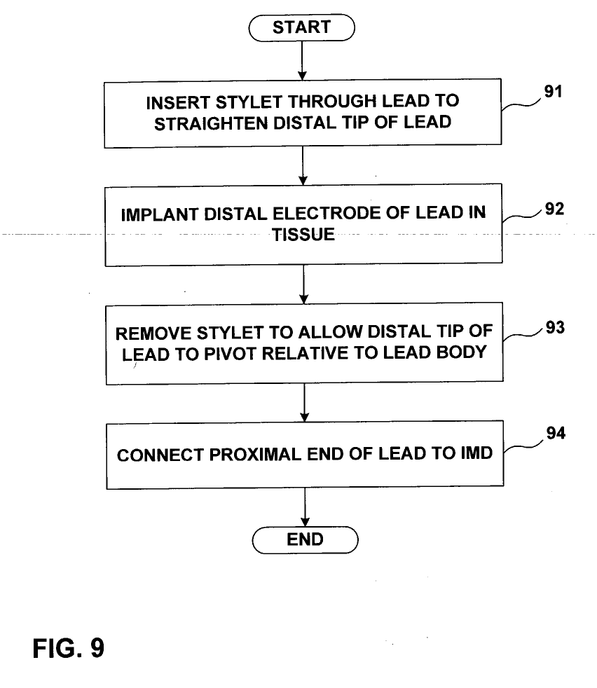

FIG. 9 is a flow diagram illustrating a procedure for implanting medial lead that includes a pivot element in accordance with the invention. As shown in FIG. 9, a stylet 73 is inserted through a lumen of lead 72 to straighten the distal tip of lead 72 (91). In particular, stylet 73 passes through lead body 75 and pivot element 78 to stabilize pivot element 78 and inhibit movement or lateral motion of electrode 71 relative to lead body 75. With the distal tip of lead 72 straightened by stylet 73, a physician implants electrode 71 within tissue 79 (92). For example, the physician can use a guiding catheter, or can simply use the stylet to guide lead 72 and position electrode 71 adjacent tissue 79. Fluoroscopic imaging techniques may also be used. In any case, once electrode 71 is positioned adjacent tissue 79, the physician rotates lead 72 to screw electrode 71 into tissue 79 and thereby anchor electrode 71 in tissue 79. However, if the pivot element comprises a ball joint, rather than a hinge element or a double hinge element, stylet 73 may need to interact with the distal most portion of lead 72 to ensure that rotation of lead 72 also causes rotation of electrode 71 such that electrode 71 screws into tissue 79.

Next, the physician removes stylet 73 from the lumen of lead 72 to allow the distal tip of lead to pivot relative to lead body 75 (93). In other words, when stylet 73 is removed, electrode 71 pivots relative to lead body 75 along an arc defined by pivot element 78. Again, such pivoting can reduce stress in tissue 79. Moreover, movement of lead body 75 within the patient, e.g., caused by patient movement, may cause additional pivoting of pivot element 78. This additional pivoting can further reduce stress to tissue 79 that would otherwise result from such movement of lead body 75. Once electrode 71 is properly implanted in tissue 79, a proximal end of lead body 75 can be coupled to an IMD (94), such as a pacemaker device, a non-cardiac implantable pulse generator (IPG), a sensing device, or the like. In accordance with the invention, the stylet can also be reinserted to facilitate removal of electrode 71 from tissue 79. Alternatively, lead 72 allows for removal of electrode 71 from tissue 79 (if desired) without the reinsertion or use of the stylet.

A number of embodiments of medical lead designs have been disclosed which can facilitate lead implantation within tissue for reduced stress on the tissue. One skilled in the art will appreciate that the present invention can be practiced with embodiments other than those disclosed. For example, although many details of the invention have been described in the context of lead implantation in the heart, the invention can find application in implantation in a wide variety of other locations, including other cardiac locations or other non-cardiac locations.

A number of examples of pivot elements have been described for use in medical leads according to the invention, including hinge elements, ball joints and double-hinge elements. Other types of pivot elements, however, could also be used in accordance with the invention. Also, although fixation electrodes in the form of helical elements have been specifically used to illustrate the invention, numerous other types of fixation electrodes could also be used. The disclosed embodiments are presented for purposes of illustration and not limitation, and the present invention is limited only by the claims that follow.

What is claimed is:

1. A medical lead comprising:
a lead body that extends from a proximal end to a distal end;
a pivot element coupled to the distal end of the lead body; and
an electrode coupled to the pivot element.

2. The medical lead of claim 1, wherein the pivot element allows for lateral movement of the electrode relative to the lead body in a two-dimensional plane.

3. The medical lead of claim 1, wherein the pivot element allows for three-dimensional movement of the electrode relative to the lead body.

4. The medical lead of claim 1, wherein the pivot element comprises a hinge element that allows for two-dimensional movement of the electrode relative to the lead body along an arc defined by the hinge-joint.

5. The medical lead of claim 1, wherein the pivot element comprises a ball-joint element that allows for three-dimensional movement of the electrode relative to the lead body.

6. The medical lead of claim 1, wherein the pivot element comprises a double-hinge element that allows for three-dimensional movement of the electrode relative to the lead body.

7. The medical lead of claim 1, wherein the electrode defines a tissue fixation structure to facilitate fixation of the electrode to tissue.

8. The medical lead of claim 1, wherein the electrode defines a helical element to facilitate fixation of the electrode to tissue.

9. The medical lead of claim 1, wherein the lead body and the pivot element are formed to define a lumen sized to permit insertion of a stylet into the medical lead.

10. The medical lead of claim 1, further comprising an electrical contact element in proximity to the proximal end and electrically coupled to the electrode via one or more conductive elements that extend along the lead body.

11. A system comprising:
an implantable medical device; and
an implantable lead including:
a lead body that extends from a proximal end to a distal end, the proximal end being coupled to the implantable medical device;
a pivot element coupled to the distal end of the lead body; and
an electrode coupled to the pivot element.

12. The system of claim 11, wherein the implantable device comprises a pacemaker device.

13. The system of claim 11, wherein the pivot element allows for lateral movement of the electrode relative to the lead body in a two-dimensional plane.

14. The system of claim 11, wherein the pivot element allows for three-dimensional movement of the electrode relative to the lead body.

15. The system of claim 11, wherein the pivot element comprises a hinge element that allows for two-dimensional movement of the electrode relative to the lead body along an arc defined by the hinge-joint.

16. The system of claim 11, wherein the pivot element comprises a ball-joint element that allows for three-dimensional movement of the electrode relative to the lead body.

17. The system of claim 11, wherein the pivot element comprises a double-hinge element that allows for three-dimensional movement of the electrode relative to the lead body.

18. The system of claim 11, wherein the electrode defines a tissue fixation structure to facilitate fixation of the electrode to tissue.

19. The system of claim 11, wherein the electrode defines a helical element to facilitate fixation of the electrode to tissue.

20. The system of claim 11, wherein the lead body and the pivot element are formed to define a lumen sized to permit insertion of a stylet into the medical lead prior to connecting the proximal end of the lead body to the implantable medical device.

21. The system of claim 11, the medical lead further including an electrical contact element in proximity to the proximal end and electrically coupled to the electrode via one or more conductive elements that extend along the lead body, wherein the electrical contact element is electrically coupled to the implantable medical device.

22. A system comprising:
a medical lead including a lead body that extends from a proximal end to a distal end, a pivot element coupled to the distal end of the lead body, and an electrode coupled to the pivot element, wherein the lead body and the pivot element are formed to define a lumen through the medical lead; and
a stylet inserted through the lumen of the lead body and the pivot element to inhibit pivoting of the pivot element.

23. The system of claim 22, wherein the pivot element comprises a hinge element that allows for two-dimensional movement of the electrode relative to the lead body along an arc defined by the hinge-joint when the stylet is not inserted through the lumen of the lead body and the pivot element.

24. The system of claim 22, wherein the pivot element comprises a ball-joint element that allows for three-dimensional movement of the electrode relative to the lead body when the stylet is not inserted through the lumen of the lead body and the pivot element.

25. The system of claim 22, wherein the pivot element comprises a double-hinge element that allows for three-dimensional movement of the electrode relative to the lead body when the stylet is not inserted through the lumen of the lead body and the pivot element.

26. The system of claim 22, wherein the electrode defines a helical element to facilitate fixation of the electrode to tissue.

27. A method comprising:
inserting a stylet through a lumen of a medical lead to inhibit pivoting of a pivot element of the medical lead;
implanting a distal electrode of the medical lead in tissue; and
removing the stylet to allow pivoting of the pivot element of the medical lead.

28. The method of claim 27, wherein the pivot element couples the electrode to a body of the medical lead.

29. The method of claim 27, wherein the pivot element allows for lateral movement of the electrode relative to the lead body in a two-dimensional plane following removal of the stylet.

30. The method of claim 27, wherein the pivot element allows for three-dimensional movement of the electrode relative to the lead body following removal of the stylet.
